(19) United States
(12) Patent Application Publication
(10) Pub. No.: US 2012/0135491 A1
(43) Pub. Date: May 31, 2012

(54) METHOD FOR PRODUCING BIOGAS OR SEWAGE GAS

(75) Inventor: Lothar Guenter, Geretsried (DE)

(73) Assignee: DGE Dr.-ING GUENTHER ENGINEERING GMBH, Wittenberg (DE)

(21) Appl. No.: 13/388,710

(22) PCT Filed: Aug. 2, 2010
(86) PCT No.: PCT/EP10/04710

§ 371 Date: Feb. 3, 2012

(30) Foreign Application Priority Data
Aug. 3, 2009 (DE) 10 2009 035 875.7

(51) Int. Cl. C12P 5/02; C12P 1/00

(52) U.S. Cl. 435/167; 435/41

(57) ABSTRACT

The invention relates to a method for producing biogas or sewage gas by a multi-stage anaerobic reaction of biomass and/or sludge. Considering the disadvantages of the known prior art, a method is to be provided that leads to a higher yield of raw gas or biogas and a higher content of methane in the raw gas and enables an economically improved operating method. To this end, the reaction is carried out in the first fermentation stage (F1) while maintaining a TS content of 3 to 8% and a volume load of 1 to 3 kg OTS/m³ d. In the second fermentation stage (F2), a further reaction of the solid matter phase is carried out while maintaining a TS content of 8 to 40% and a volume load of over 2 kg OTS/ m³ d. In the second fermentation stage, the fermentation substrate is set to a TS content that is higher than the TS content of the first stage. In both fermentation stages, the reaction is carried out in the range from slightly acidic to neutral (pH value 6.5 to 8). The biogas obtained in the fermentation stages (F1, F2) is combined or removed separately and is subjected to further purification.

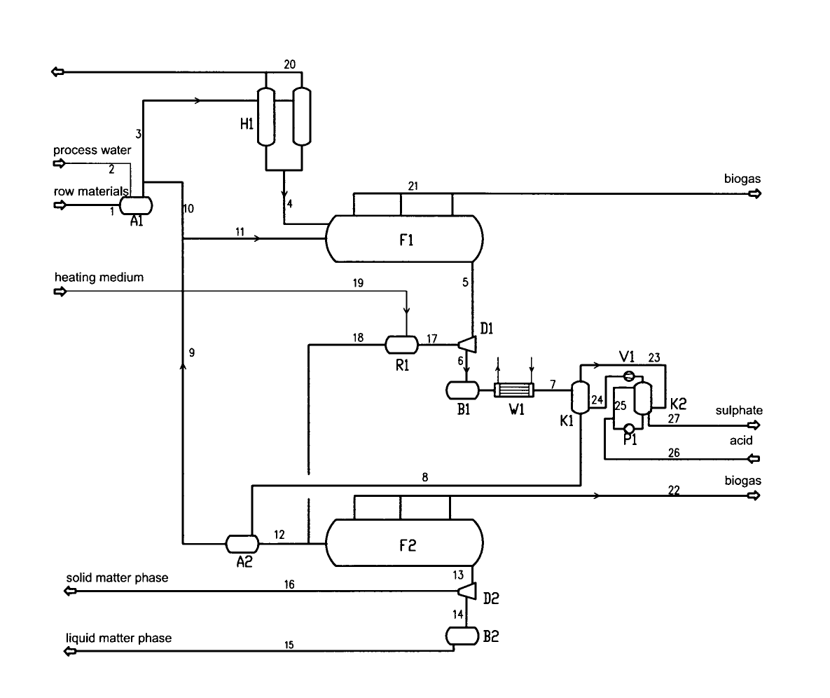

The invention relates to a method of producing biogas or sewage gas by a multi-stage anaerobic reaction of biomass and/or sludge.

Biogas is produced in a manner known per se in one or a plurality of reactors or fermenters which can be operated mesophilically (at temperatures below 45° C.) or thermophilically (at temperatures from 45° C. to 80° C.).

Organic materials in the form for example of agricultural fertilisers (slurry, manure), renewable raw materials and biological waste materials are used as biomass.

Sludge is not suitable for an anaerobic reaction unless it is derived from organically contaminated waste or process water with a COD content of over 5,000 mg/1 and is therefore a biomass that can be converted to biogas gas anaerobically. COD stands for chemical oxygen demand which is determined as part of a COD measurement and is a measure of the amount of oxygen required by the chemical digestion/purification processes taking place in the wastewater. Biogas is produced or manufactured by different biodegradation processes which take place during the reaction as hydrolysis, acidogenesis, acetogenesis and methanogenesis. The degradation processes caused by bacteria can take place under aerobic or anaerobic conditions. The most widely used method is the wet fermentation method in which the dry substance content TS is <15% and the water content is >85%.

In practice biogases with a methane content of up to 65% can be produced dependent on the raw materials used and the processes operated in the biogas plant.

Biogas is used for example for heating purposes for combined heat and power plants or as an energy source for feeding into natural gas networks.

The CO₂ contained in the biogas, as well as other impurities, particularly hydrogen sulphide, nitrogen and ammonia, must be removed in order to produce methane gas of a high quality suitable for further processing.

The purification or reprocessing of biogas is a technologically complicated process involving a high level of expenditure for the apparatus required.

The costs of the subsequent purification or reprocessing to methane gas decrease as the methane content in the biogas produced increases.

Solutions are therefore already known for modifying the process of biogas production in such a way as to produce a biogas with the highest possible methane content.

In DE 103 16 680 A1 it is proposed to load a prefermentation reactor with a nutrient solution and to continue feeding an untreated biomass into this reactor until a pH value of 4.3 to 4.8 has been established. The prefermented biomass is then fed to the main reactor at a quantity sufficient to establish and maintain a constant pH value of 6.7 to 7.7. Digested material from the main reactor is fed to the prefermentation reactor as a nutrient solution. New biomass can be continuously added to this prefermentation reactor. One part of the product of the prefermentation reactor is fed to the main reactor and vice versa in amounts that should be approximately equal. Both reactions are circulated in the circuit, a process in which the pH value is the key control variable. The disadvantage of this method is that the bacteria for hydrolysis, acidogenesis, acetogenesis and methanogenesis are mixed. The biogas produced in the prefermentation reactor has a very low methane content of 5 to 20% so that it can be used only by mixing it with biogas from the main reactor.

A method is described in DE 10 2007 037 202 A1 of converting biomass to biogas which is carried out in fermenters under anaerobic conditions. Renewable raw materials together with liquid and other starting materials required for methanogenesis are fed to the first fermenter and undergo a fermentation process. The digestate is then separated into a solid-liquid-phase and the solid matter phase undergoes a thermo pressure hydrolysis at temperatures of at least 170° C. and pressures of at least 1 MPa. The solid matter phase treated in this manner can either be returned to the first fermenter or fed to a second fermenter for a further fermentation process. The process step of subjecting the separated solid matter phase to a thermo pressure hydrolysis is complex and cost-intensive.

In DE 10 2007 000 834 A1 it is proposed to wash and comminute ensilaged renewable raw materials, to remove a part of the scrubbing water and subject these materials to hydrolysis. The hydrolysis products then undergo further treatment in fermenters in a manner known per se in order to produce biogas. The use of ensilaged renewable raw materials is disadvantageous on ecological grounds. The washing and comminuting steps required are energy and cost-intensive.

A disadvantage common to all the known methods is that the yields of methane produced in converting biomass and/or sludge to biogas or sewage gas are too low.

The objective of the invention is to devise a method of producing biogas or sewage gas which produces a higher yield of raw or biogas as well as a raw gas with a higher methane content and which enables a biogas plant to be operated more economically.

This objective is achieved according to the invention by means of the features specified in claim 1. Advantageous developments of the method are the subject matter of claims 2 to 13.

The reaction of the biomass in both fermentation stages is carried out in the range from slightly acidic to neutral (pH value 6.5 to 8). In the first fermentation stage the reaction is carried out while maintaining a TS content of 3 to 8% and a volume load of 1 to 3 kg OTS/m³. A further reaction of the solid matter phase of the fermentation substrate from the first fermentation stage is carried out in the second fermentation stage while maintaining a TS content of 8 to 40% and a volume load of over 2 kg OTS/m³. In this process it is crucial that the TS content of the fermentation substrate in the second fermentation stage is set at a value that is greater—preferably 1.5 to 20 times greater—than the TS content of the first stage.

In the first fermentation stage the processes of hydrolysis, acidogenesis and acetogenesis run in parallel. Hydrolysis takes place at a pH value in the range from slightly acidic to neutral. This represents a departure from the previous usual practice (pH 4.5 to 6). In combination with the low reactor loading less acidogenesis occurs which accelerates methanogenesis. In the first fermentation stage a biogas with a high CO₂ content is produced. As this is being continuously led off and purified, a biogas with a higher methane content is produced in the fermenter. Surprisingly, it was found that a higher yield of biogas with a higher methane content can be obtained by maintaining the above-named values of the process parameters of pH value, TS content and volume load as well as a higher TS content in the second fermentation stage.

The first fermentation stage is carried out as a wet fermentation. The second fermentation stage can also be operated as a dry fermentation process. The optimum method in each case also depends primarily on the composition of the biomass.

The above results can be improved by pretreating the biomass in a pressureless manner at temperatures of 25 to 60° C. in a hydrolysis stage upstream of the first fermentation stage. Hydrolysis is carried out here at a pH value of 5 to 8. The retention period of the biomass in the hydrolysis stage should be 3 to 8 days, preferably 4 to 6 days. The retention time in the hydrolysis stage can also be determined by the H₂S concentration measured in the biogas that is drawn off. To that end the H₂S concentration is measured and evaluated continuously or at specific intervals.

The curve progression of the measured values (H₂S concentration in ppm and time elapsed in hours) recorded as a curve chart clearly shows a peak (increased H₂S concentration) after which the values slowly fall again. A lower limit value for the H₂S concentration is set dependent on the raw materials used and the actual hydrolysis conditions. If this limit value is reached after the peak has been reached, hydrolysis is terminated.

Different H₂S concentrations and curve progressions are produced dependent on the raw materials used and the composition of the biomass.

The gas mixture (biogas) produced during hydrolysis containing CO₂ and with a high H₂S concentration is drawn off and purified, producing a biogas with a methane content that is 3 to 10 times higher. The gas can be purified by a water scrubbing process carried out in a pressureless manner. The hydrolysis gas or biogas can be further purified up to a methane content of 50% by volume or higher. The carbon dioxide removed in the process is utilised for other purposes. A further advantage of an upstream hydrolysis stage lies in the fact that the H₂S content is reduced in the downstream fermentation stages. Hydrolysis is then prevented or can take place only to a greatly diminished degree in the subsequent fermentation stages. Under these conditions biocultures develop which have a very advantageous effect on methanogenesis.

The biogas drawn off from the hydrolysis stage and the two fermentation stages is purified and subsequently combined to form one gas stream for further utilisation. Alternatively, the gas streams can also be purified individually or after they have been combined. This depends primarily on the composition of the biogas contained in the individual stages. The costs of purification involved, which should be kept as low as possible, should also be taken into account here.

It may also be possible to integrate an additional hydrolysis stage into the second fermentation stage. If necessary, additional small amounts of raw materials or biomass may also be fed in during the second fermentation stage.

Methane yields of up to 80% and up to about 20% can be achieved in the first and second fermentation stage respectively dependent on the starting material used. This depends however on the type and composition of the raw materials used and the methane concentration required for the subsequent utilisation of the gas.

An additional advantage is achieved if a partial quantity at least of the liquid phase removed from the digestate from the first fermentation stage is fed to a stripping stage in which the ammonia contained in the liquid phase is removed. The fermentation water purified in this way with an ammonia content of under 2g/l, preferably as low as 0.5 mg/1, is for example stored temporarily. The purified fermentation water can then be used for further batches of biomass or fermentation substrate. This improves the biology in the starting material compared with the method of adding fresh water, enabling a biogas with a higher methane content to be obtained.

The solid matter phase removed from the digestate from the first fermentation stage can be mixed with the purified liquid phase (fermentation water) before being fed to the second fermenter or can be thermally treated in an interposed reactor at temperatures of up to 180° C. and a pressure of up to 10 bar, if necessary with the addition of additives acting as acids or alkalis. The contaminated stripping gas can be treated in a downstream scrubbing stage by means of an acidic scrubbing solution. This converts the ammonia contained in the gas stream into ammonium sulphate or phosphorus sulphate or other salts. The sulphate removed from the circuit can be used as an agricultural fertiliser.

The invention is now to be described by means of some examples.

The associated drawing depicts a plant for carrying out the method. The plant is to be explained in more detail using the examples below.

### EXAMPLE 1

The following raw materials are used as biomass to produce biogas:

Bovine slurry with a TS content of 6%, manure with a TS content of 25%, maize with a TS content of 32% and grass silage with a TS content of 30%.

The raw materials (1512 kg bovine slurry, 302 kg manure, 116 kg maize and 349 kg grass silage) are conveyed via the line 1 into a mixing tank A1 (1.96 m³/h). The mixture has a TS content of 12.2% and is adjusted in the mixing tank by the unpurified fermentation water already in the tank (1.6 m³/h) so as to produce a TS content of 6% in the mixing tank. Fermentation or process water is fed in via the line 2. No additional additives are added to the fermentation substrate which is conveyed continuously via the line 11 into the first fermenter F1 at an amount of 3.57 m³/h after a retention time of about 1 hour in the mixing tank. This fermenter is operated mesophilically (at a temperature of 38° C.) and at a volume load OTS of 1 kg/m³d. The volume load is continuously monitored by suitable measuring methods and set to the above-mentioned value by feeding in fermentation substrate if the value is undershot or exceeded. Moreover, the pH value, which is set to a value of 7.5 to 7.8, is continuously monitored. The plant is operated in the first fermenter F1 in the neutral range. The fermentation substrate is maintained at the above-mentioned temperature by the fermenter heater or cooler. The fermenter is insulated in a usual manner per se and is fitted with an agitator for mixing the fermentation substrate.

A superimposed hydrolysis and acidogenesis phase as well as methanogenesis takes place in the first fermenter F1 under the conditions referred to above. The hydrogen sulphide content in the biogas is reduced by the metered addition of air or oxygen or iron salts.

A biogas with the following composition is produced during the biological reaction of the fermentation substrate in the first fermenter:

| | | |
|---|---|---|
| CH₄ | 55% | by volume |
| CO₂ | 41% | by volume |
| H₂O | 3.5% | by volume |
| H₂S | 60 | ppm |
| NH₃ | 35 | ppm |

On average 96 Nm³/h of gas is produced in the first fermenter F1. The biogas produced is led off via the line 21 and mixed with biogas from the fermenter F2. The mixture (biogas) is subsequently purified and utilised.

The reaction of the fermentation substrate in the first fermenter F1 is terminated after a retention time of the fermentation substrate of 20 days.

The fermentation substrate is drawn off from the first fermenter F1 via line 5 and fed to a separating device D1 (separator) to be separated into a liquid and a solid matter phase. The solid matter phase (water content of 30 up to 70% by weight) can be fed via the lines 17, 18 either to a reactor R1 or directly to the second fermenter F2. The reactor R1 is heatable, with the heating medium fed in via the line 19. The separated liquid phase (fermentation water) reaches a tank B1 via line 6 and is stored temporarily for further use. A partial quantity can be returned to the mixing tank or the first fermenter.

The solid matter phase of the fermentation substrate is set to a TS content of 9% in the second fermenter F2. The plant is operated in the second fermenter F2 under mesophilic conditions (at a temperature of 38 to 42° C.) and with a volume load OTS of 2.2 kg/m³d. The biological reaction of the fermentation substrate takes place under exclusion of air or oxygen and at pH values of 7.5 to 7.8. The retention time in the second fermenter F2 is approximately 40 to 60 days. Metered small amounts of iron salts can be added to reduce the formation of hydrogen sulphide.

18 Nm₃/h of biogas with the following composition is produced during the reaction in the second fermenter F2:

| | | |
|---|---|---|
| CH₄ | 54% | by volume |
| CO₂ | 43% | by volume |
| H₂O | 3% | by volume |
| H₂S | 40 | ppm |
| NH₃ | 42 | ppm |

The biogas is led off via the line 22, combined with the biogas from the first fermenter and purified for further use.

A total of approximately 114 Nm³/h of biogas with a methane content of 54.8% by volume is produced in both fermenters F1 and F2. The amount of methane produced is 62.5 m³/h and is therefore 35% greater than that produced by known conventional methods.

The different conditions with regard to the TS content and the volume load for the reaction of the fermentation substrate in the first and second fermenter lead to a higher biogas yield and an increased methane content. The reaction is in each case carried out in the neutral range,

### EXAMPLE 2

The biomass in mixing tank A1 is mixed under the same conditions as described in example 1. No additional additives are added to the fermentation substrate. The fermentation substrate (amount 3.57 m³/h) is conveyed via the line 3 into a hydrolysis tank H1 after a retention time of about 1 hour in the mixing tank A1 and pretreated in an hydrolysis stage upstream of the fermentation process in a pressureless manner at temperatures of 35° C. and a pH value of 6.5 to 7.5. The retention time for the hydrolysis and acidogenesis reaction in the hydrolysis tank H1 is 4 days. The hydrolysis reactor is operated in batches. As can be seen from the drawing two hydrolysis tanks are envisaged.

Unlike other known hydrolysis stages hydrolysis takes place in the neutral and not in the acidic range. Under these conditions a gas containing CO₂ and with small traces of hydrogen and methane and a high concentration of hydrogen sulphide is produced in the hydrolysis process.

The biogas (32 Nm³/h) escaping via line 20 has the following composition:

| CO₂ | 60 to 85% | by volume |
|---|---|---|
| H₂ | up to 5% | by volume |
| CH₄ | up to 10% | by volume |
| H₂S | 800 | ppm (mean value) |

| | |
|---|---|
| Remainder | air |

During hydrolysis the H₂S concentration increases after approximately 52 hours to a value of 2850 ppm (peak) after which it slowly falls again. The H₂S concentration is measured at intervals of 60 minutes. The H₂S concentration falls to a value of 420 ppm and the hydrolysis process is terminated after approximately a further 43 hours.

The biogas drawn off from the hydrolysis stage H1 is treated in a purification stage which is not illustrated. This stage is operated for example as a water scrubbing process carried out in a pressureless manner in which the hydrolysis gas or biogas with a methane content of 50% by volume is purified at an amount of approximately or up to 4 Nm₃/h of methane. The carbon dioxide removed is used for other purposes. The fermentation substrate from the hydrolysis stage H1 is continuously fed to the fermenter F1 via the line 4 and treated in said fermenter under the same conditions as described in Example 1.

Because of the upstream hydrolysis the metered addition of air, oxygen or iron salts to reduce the hydrogen sulphide in the biogas can be reduced by approximately 80% as most of the hydrogen sulphide has already been removed in the hydrolysis stage.

A biogas with the following composition is produced during the biological reaction of the fermentation substrate in the first fermenter F1:

| | | |
|---|---|---|
| CH₄ | 72.26% | by volume |
| CO₂ | 24.24% | by volume |
| H₂O | 3.5% | by volume |
| H₂S | 20 | ppm |
| NH₃ | 31 | ppm |

On average 73 Nm³/h of gas is produced. This biogas drawn off via the line 21 can be mixed with biogas from the second fermenter F2 or can also be used separately if required. The reaction of the fermentation substrate in the first fermenter F1 is terminated after a retention time of 20 days.

The fermentation substrate drawn off from the first fermenter F1 via the line 5 is separated into a liquid phase and a solid matter phase (water content of 30 up to 70% by weight) in a similar way to that described in Example 1.

The solid matter phase of the fermentation substrate is treated in the second fermenter F2 under the same conditions as described in Example 1.

18 Nm₃/h of biogas with the following composition is produced during the reaction in the second fermenter F2:

| | | |
|---|---|---|
| CH₄ | 54% | by volume |
| CO₂ | 43% | by volume |
| H₂O | 3.5% | by volume |
| H₂S | 40 | ppm |
| NH₃ | 39 | ppm |

The biogas is led off via the line 22 for further utilisation.

A total of approximately 95 Nm³/h of biogas with a methane content of 67.9% by volume is produced in the hydrolysis stage H1 and the two fermenters F1 and F2. The amount of methane produced is 64.5 m³/h and is therefore 39.3% higher than that produced by known conventional methods.

A biogas stream with a methane content of 72.26% by volume is produced in the fermenter F1. The methane content can be increased to over 80% by volume by extending the retention time in the hydrolysis stage by approximately 2 to 3 days.

### EXAMPLE 3

The only difference between Example 3 and Example 2 is that Example 3 includes a stripping stage K1 interposed downstream of the first fermentation stage F1 in which ammonia is removed from the heated fermentation water down to a concentration of 0.5 mg/l by means of stripping gas under increased pressure preferably between 10 to 100 mbar.

The liquid phase which has been removed (fermentation water) is fed via line 6 to the tank B1. A partial stream (approximately 50%) is branched off from said tank via the line 7 and heated in a downstream heat exchanger W1 up to approximately 50 to 70° C. with the pH set if necessary to a value over 8. The heated partial stream is then fed to a stripping column K1 in which stripping gas is increased to a pressure of approximately 10 to 20 mbar by the compressor V1 integrated in stripping gas lines 23, 24, and the ammonia contained in the fermentation water is stripped out. The contaminated circulation or stripping gas is treated in a scrubbing column K2 with an acidic scrubbing solution. The scrubbing solution conducted in the line 25 is circulated in the circuit by the pump P1.

On contact with the acidic scrubbing solution the ammonia contained in the stripping gas is converted to ammonium sulphate or phosphorus sulphate. The concentration of sulphate is set to about 10 to 30% by weight, with the acid metered via the line 26. The sulphate formed is removed via the line 27 from the circuit at the base of the scrubbing column K2 for use as a fertiliser.

The ammonium concentration in the fermentation water is reduced from 2 to 0.5 mg/l by the stripping process.

The fermentation water, which contains almost no ammonia, can then be re-used in the biological process for setting the TS content of the biomass used during mixing. This has an advantageous effect on the biology of the reaction of the fermentation substrate.

Where purified fermentation water is used in the mixing phase, a biogas with the following composition is produced during the biological reaction of the fermentation substrate in the first fermenter F1:

| | | |
|---|---|---|
| CH₄ | 72.61% | by volume |
| CO₂ | 23.89% | by volume |
| H₂O | 3.50% | by volume |
| H₂S | 20 | ppm |
| NH₃ | 8 | ppm |

On average 75 Nm³/h of gas is produced.

18 Nm³/h of biogas with the following composition is produced during the reaction in the second fermenter F2.

| | | |
|---|---|---|
| CH₄ | 54% | by volume |
| CO₂ | 43% | by volume |
| H₂O | 3% | by volume |
| H₂S | 40 | ppm |
| NH₃ | 6 | ppm |

A total of approximately 97 Nm³/h of biogas with a methane content of 68.2% by volume is produced in the hydrolysis stage and the two fermenters F1 and F2. The amount of methane produced amounts to 66.2 m³/h. The increase in the methane yield compared with Example 2 is attributable to the lower ammonia content in the fermentation water added.

The digestate residue treated in the reactor R1 can be fed directly to the second fermenter F2. A second mixing tank A2 to which purified fermentation water can be fed via the line 8 can also be positioned upstream of said second fermenter. Purified fermentation water can be fed to both the first mixing tank A1 and the second fermenter F2 via the lines 9, 10, 12 connected to this second mixing tank.

On completion of the reaction in the second fermenter F2 the digestate residue is fed via the line 13 to a second separator D2. The solid matter phase is led off via the line 16 and the liquid phase reaches a second receiver tank B2 via the line 14 and can be led off from said tank via the line 15.

### EXAMPLE 4

This example differs from Example 3 in that the hydrolysis stage continues to be operated until the H₂S concentration in the hydrolysis gas or biogas which has been led off has fallen to a lower limit value of 240 ppm after reaching a peak. This value was reached after approximately 142 hours, thereby achieving a higher yield of biogas. Moreover, the proportion of methane in the biogas increases as well as a result. 43 Nm³/h of biogas, which contains CO₂ and has an identical composition to the biogas in Example 3, is produced in the hydrolysis process.

The biogas from the hydrolysis stage is treated by water scrubbing carried out in a pressureless manner in which the hydrolysis gas or biogas with a methane content of 50% by volume is purified at an amount of approximately or up to 8 Nm₃/h of methane.

During the biological reaction of the fermentation substrate in the first fermenter F1 a biogas with the following composition is produced:

| | | |
|---|---|---|
| CH₄ | 81.97% | by volume |
| CO₂ | 14.53% | by volume |
| H₂O | 3.50% | by volume |
| H₂S | 17 | ppm |
| NH₃ | 8 | ppm |

On average 64 Nm³/h of gas is produced.

During the subsequent reaction in the second fermenter F2 18 Nm³/h of biogas with the following composition is produced:

| | | |
|---|---|---|
| CH₄ | 54% | by volume |
| CO₂ | 43% | by volume |
| H₂O | 3% | by volume |
| H₂S | 40 | ppm |
| NH₃ | 6 | ppm |

A total of 90 Nm₃/h of biogas with a methane content of 73.6% by volume is produced in the hydrolysis stage and the two fermentation stages F1 and F2. The amount of methane produced is 66.2 m³/h and therefore exceeds the amounts produced by the known conventional methods by 43.0%.

A biogas stream with a methane content of 81.971% by volume is produced in the fermenter 1. Because of its high methane content the biogas can even be fed to a gas supply network after fine desulphurisation and drying has been carried out.

In practice the respective pH values can be set by feeding in raw materials, fermentation water or fresh water as well as by the TS content.

### Comparative Results:

The following yields of biogas and methane produced from the starting materials set out below are given in the technical literature, “Biogas basic data Germany, status: August 2007”, German Agency for Renewable Resources (FNR), 18276 Gülzow:

| | | |
|---|---|---|
| 13,000 t bovine slurry | 325,000 m³ biogas | 195,000 m³ methane |
| (TS content 6%) | | |
| 2,600 t bovine manure | 117,000 m³ biogas | 70,200 m³ methane |
| (TS content 25%) | | |
| 1,000 t maize silage | 202,000 m³ biogas | 105,040 m³ methane |
| (TS content 30%) | | |
| 300 t grass silage | 51,600 m³ biogas | 27,864 m³ methane |
| (TS content 30%) | | |
| Total | 695,600 m³ biogas | 398,104 m³ methane |

80.9 Nm³/h of biogas with a methane content of 57.23% by volume is therefore produced in a continuous operation of a conventional biogas plant over a period of 8,600 hours. The amount of methane produced is 46.3 Nm³/h.

1. A method of producing biogas or sewage gas, the method comprising:
(a) fermenting a starting material to form a digestate;
(b) separating the digestate into a solid matter phase and a liquid phase;
(c) treating the solid matter phase to form a treated solid matter phase;
(d) fermenting the treated solid matter phase;
(d) combining or removing separately gases obtained in the fermenting (a) and the fermenting (d) to obtain at least one initial gas; and
(e) purifying the at least one initial gas to form a biogas or sewage gas, wherein:
the starting material is biomass, sludge, or both;
the fermenting (a) and (d) involve anaerobic wet fermentation;
the fermenting (a) is carried out while maintaining a TS content of 3 to 8% and a volume load of 1 to 3 kg OTS/m³ d;
the fermenting (d) is carried out while maintaining a TS content of 8 to 40% and a volume load of over 2 kg OTS/m³ d;
a fermentation substrate in the fermenting (d) is set to a TS content that is higher than a TS content of the fermenting (a); and
the fermenting (a) and (d) are is carried out in a pH range from slightly acidic to neutral, such that a pH value is 6.5 to 8.

2. The method of claim 1, further comprising:
(H1) pretreating the starting material in a pressureless manner, in a hydrolysis stage (H1) before the fermenting (a), at temperatures of 25 to 60° C. and at a pH value of 5 to 8.

3. The method of claim 2, wherein
a retention period of the starting material in the hydrolysis stage (H1) is 3 to 8 days,
the pretreating (H1) produces producing a gas mixture comprising CO₂ and a high concentration of H₂S, and
the gas mixture is drawn off and purified.

4. The method of claim 3, wherein
the retention period is controlled by an H₂S concentration measured in the gas mixture that is drawn off,
a lower limit value is set dependent on a composition of raw materials and hydrolysis conditions, and
the hydrolysis (H1) is terminated after a peak has been reached and the H₂S concentration has subsequently fallen to the lower limit value.

5. The method of claim 2, further comprising:
(H2) purifying a gas drawn off from the hydrolysis stage (H1), thereby increasing a methane content of the biogas or sewage gas by 3 to 5 times.

6. The method of claim 2, wherein initial gases drawn off from the hydrolysis stage (H1) and the fermenting (a) and (d) are purified and subsequently combined to form one gas stream.

7. The method of claim 1, further comprising:
(K1) removing ammonia contained in the liquid phase by feeding at least a partial amount of the liquid phase to a stripping stage (K1) in which the ammonia is removed to form a purified liquid phase; and
(K3) temporarily storing the purified liquid phase.

8. The method of claim 7, further comprising:
(K4) mixing the solid matter phase with the purified liquid phase to form a resulting solid/liquid mixture; and
(K5) feeding the resulting solid/liquid mixture to the fermenting (d).

9. The method of claim 1, further comprising:
(A1) pretreating the starting material in a mixing stage (A1) with the addition of fermentation water, having a reduced ammonia content, from the method.

10. The method of claim 1, further comprising:
(A2) pretreating the solid matter phase in a second mixing stage (A2) with the addition of fermentation water, having a reduced ammonia content, from the method.

11. The method of claim 1, further comprising;
(R1) thermally treating the solid matter phase at temperatures of up to 180° C. and a pressure of up to 10 bar with the addition of at least one additive acting as an acid or an alkali.

12. The method of claim 7, wherein the ammonia, down to a concentration of 0.5 mg/l, is removed from heated fermentation water in the stripping stage (K1) with a stripping gas under increased pressure.

13. The method of claim 12, further comprising
(K2) treating contaminated stripping gas in a scrubbing stage (K2) with an acidic scrubbing solution, wherein the ammonia is converted to ammonium or phosphorus sulphate or at least one other salt and is removed.

14. The method of claim 2, wherein
a retention period is controlled by an H₂S concentration measured in a gas mixture that is drawn off from the hydrolysis stage (H1),
a lower limit value is set dependent on a composition of raw materials and hydrolysis conditions, and
the hydrolysis (H1) is terminated after a peak has been reached and the H₂S concentration has subsequently fallen to the lower limit value.
